(19) United States
(12) Patent Application Publication
(10) Pub. No.: US 2015/0118296 A1
(43) Pub. Date: Apr. 30, 2015

(54) CONTROLLED RELEASE BUDESONIDE COMPOSITIONS

(71) Applicant: CADILA HEALTHCARE LIMITED, Ahmedabad (IN)

(72) Inventors: Shushrut Krishnaji Kulkarni, Ahmedabad (IN); Ajaykumar Handa, Ahmedabad (IN); Pushpendra Pratap Singh, Ahmedabad (IN); Ankur Paresh Shah, Ahmedabad (IN)

(21) Appl. No.: 14/520,634

(22) Filed: Oct. 22, 2014

(30) Foreign Application Priority Data
Oct. 25, 2013 (IN) 3373/MUM/2013

(51) Int. Cl. A61K 9/28; A61K 9/20; A61K 31/58

(52) U.S. Cl. CPC A61K 9/28; A61K 31/58; A61K 9/2013; A61K 9/2054
USPC 424/452; 424/465; 514/174

(57) ABSTRACT

The present invention relates to controlled release pharmaceutical compositions comprising budesonide. The invention also relates to processes for the preparation of such compositions and using those compositions in the treatment of Inflammatory Bowel Disease and Irritable Bowel Syndrome including mild to moderate ulcerative colitis.

### FIELD OF THE INVENTION

The present invention relates to controlled release pharmaceutical compositions comprising budesonide. The invention also relates to processes for the preparation of such compositions and using those compositions in the treatment of Inflammatory Bowel Disease and Irritable Bowel Syndrome including mild to moderate ulcerative colitis.

### BACKGROUND OF THE INVENTION

Budesonide is designated chemically as (RS)-11β,16α,17,21-tetrahydroxypregna-1, 4-diene-3, 20-dione cyclic 16, 17-acetal with butyraldehyde and having a structure of the following Formula:

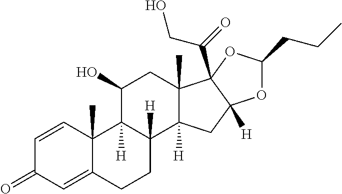

The use of glucocorticoids, in particular budesonide, is generally known for the treatment of diseases which are associated with inflammation processes. The active ingredient budesonide has also been used successfully for the treatment of Inflammatory Bowel Disease and Irritable Bowel Syndrome including mild to moderate ulcerative colitis.

The preparation of a sustained, controlled, delayed, extended or anyhow modified release form can be carried out according to different techniques:

1. The use of inert matrices, in which the main component of the matrix structure opposes some resistance to the penetration of the solvent due to the poor affinity towards aqueous fluids; such property being known as lipophilia.
2. The use of hydrophilic matrices, in which the main component of the matrix structure opposes high resistance to the progress of the solvent, in that the presence of strongly hydrophilic groups in its chains, mainly branched, remarkably increases viscosity inside the hydrated layer.
3. The use of bioerodible matrices, which are capable of being degraded by the enzymes of some biological compartment.

All the procedures listed above, however, suffer from drawbacks and imperfections.

Inert matrices, for example, generally entail non-linear, but exponential, release of the active ingredient.

Hydrophilic matrices have a linear behavior until a certain fraction of active ingredient has been released, then significantly deviate from linear release.

Bioerodible matrices involve the problem of finding the suitable enzyme or reactive to degradation. Furthermore, they frequently release in situ metabolites that are not wholly toxicologically inert.

International (PCT) Publication No. WO 95/16451 discloses a composition only formed by a hydrophilic matrix coated with a gastro-resistant film for controlling the dissolution rate of the active ingredient.

D'Haens et al., Journal of Crohns and Colitis (2010) discloses a MMX® extended release Budesonide composition. This MMX® technology teaches MMX® tablets comprising lipophilic and amphiphilic matrices dispersed in a hydrophilic matrix. The MMX® tablets are coated with a gastro-resistant coating.

International (PCT) Publication No. WO 00/76478 discloses controlled release oral compositions containing as active ingredient budesonide comprising a matrix consisting of amphiphilic compounds and lipophilic compounds with melting point below 90° C. in which the active ingredient is at least partially incorporated and an outer hydrophilic matrix in which the lipophilic-amphiphilic matrix is dispersed.

The present disclosure relates to methods for treating intestinal diseases presenting at least one inflammatory component such as inflammatory bowel disease and/or maintaining remission of intestinal diseases using budesonide controlled release compositions.

It has been found that controlled release budesonide compositions can also be prepared without using a lipophilic component. The instant invention addresses the mentioned unmet needs by providing alternate modified compositions of budesonide characterized by controlled release of budesonide.

### SUMMARY OF THE INVENTION

In one general aspect, there is provided a controlled release pharmaceutical composition of budesonide comprising:

(1) a tablet core comprising budesonide in an amount effective for the treatment of inflammatory bowel disease in the gastrointestinal tract, an amphiphilic excipient, a hydrophilic excipient and one or more pharmaceutically acceptable excipients; and
(2) a gastro-resistant coating on the tablet core.

In another general aspect, the present invention provides a controlled release pharmaceutical composition of budesonide, wherein the composition comprises about 1 mg to about 12 mg of budesonide.

In another general aspect, there is provided a controlled release pharmaceutical composition of budesonide, wherein the amphiphilic excipient comprises about 1 to 15% by weight of the composition.

In another general aspect, there is provided a controlled release pharmaceutical composition of budesonide, wherein the amphiphilic excipient is soy lecithin.

In another general aspect, there is provided a controlled release pharmaceutical composition of budesonide, wherein the hydrophilic excipient comprises about 1 to 30% by weight of the composition.

In another general aspect, there is provided a controlled release pharmaceutical composition, wherein budesonide is homogeneously dispersed in the amphiphilic excipient.

In another aspect, there is provided a controlled release pharmaceutical composition, wherein the budesonide is homogeneously dispersed in the hydrophilic excipient and in an amphiphilic excipient.

In still another general aspect, there is provided a controlled release pharmaceutical composition, wherein the composition is in the form of a tablet, minitablets, pellets, a capsule, a caplet, a sachet, beads or granules.

In further general aspect, there is provided a controlled release budesonide composition wherein the composition releases not more than about 80% of budesonide within about 8 hours in vitro.

Embodiments of the pharmaceutical composition may include one or more of the following features. For example, the pharmaceutical composition may further include one or more pharmaceutically acceptable excipients. The pharmaceutically acceptable excipients may include one or more fillers, binders, disintegrants, lubricants, glidants, antiadherents, solubilizers, sweeteners, flavors, taste-masking agents and the like.

In another aspect, the invention provides a process for the preparation of a controlled release pharmaceutical composition comprising budesonide, the process comprising the steps of:

a) mixing budesonide with an amphiphilic excipient, a hydrophilic excipient and one or more pharmaceutically acceptable excipients,
b) granulating the mixture using a solvent,
c) drying the granules obtained,
d) milling the dried granules,
e) blending the milled granules with the remaining quantity of an amphiphilic excipient and one or more pharmaceutically acceptable excipients,
f) lubricating the blend and compressing into tablets; and
g) optionally coating the tablets with a delayed release coat.

In yet another aspect, there is provided a method for the treatment of Inflammatory Bowel Disease or Irritable Bowel Syndrome including mild to moderate ulcerative colitis, comprising administering the controlled release composition of the present invention to a patient in need of such treatment.

In still another general aspect, there is provided a controlled release pharmaceutical composition of budesonide comprising:

(1) a tablet core comprising budesonide in an amount effective for treatment of inflammatory bowel disease in the gastrointestinal tract, an amphiphilic excipient, a hydrophilic excipient; and one or more pharmaceutically acceptable excipients; and
(2) a gastro-resistant coating on the tablet core,
wherein the composition exhibits no significant difference in both rate and extent of absorption of budesonide as compared to extended release composition of budesonide marketed under trade name Uceris®.

Embodiments of the pharmaceutical composition may include one or more of the following features. For example, the pharmaceutical composition may further include one or more pharmaceutically acceptable excipients. The pharmaceutically acceptable excipients may include one or more fillers, binders, disintegrants, lubricants, glidants, antiadherents, solubilizers, sweeteners, flavors, taste-masking agents and the like.

The details of one or more embodiments of the invention are set forth in the description below. Other features, objects and advantages of the invention will be apparent from the description.

### DETAILED DESCRIPTION OF THE INVENTION

The present invention provides a controlled release pharmaceutical composition of budesonide for oral administration comprising budesonide as an active ingredient, wherein the active ingredient is released from the composition at a controlled rate along a pre-determined release profile, and wherein the controlled release budesonide composition comprises a tablet core comprising an amphiphilic excipient, a hydrophilic excipient, one or more pharmaceutically acceptable excipients and a gastro-resistant film coating.

It has now surprisingly been found that a budesonide composition comprising hydrophilic excipient and an amphiphilic excipient can be prepared, thus avoiding the use of a lipophilic matrix forming excipient.

For the purposes of this invention, the term “budesonide” includes budesonide or any pharmaceutically acceptable salts or derivatives thereof, including polymorphs, hydrates, solvates or amorphous forms.

The term “controlled release” as used herein can be used synonymously with extended release, sustained release, modified release, delayed release or pulsatile release.

As used herein, unless otherwise noted, “rate of release” or “release rate” of a drug refers to the quantity of drug released from a dosage form per unit time, e.g., milligrams of drug released per hour (mg/hr) or a percentage of a total drug dose released per hour. Drug release rates for dosage forms are typically measured as an in vitro rate of drug release i.e., a quantity of drug released from the dosage form per unit time measured under appropriate conditions and in a suitable fluid.

The release rates referred to herein are determined by performing dissolution test by introducing individual tablets in a rotating basket type dissolution apparatus containing from 500 to 1000 ml of a buffered solution set to different pH conditions (pH 1 and 7.2 are the pH condition generally used in this test application), so that the pH conditions, from stomach to large intestine, should be reproduced. To simulate the human body conditions, the test is carried out at a temperature of 37° C.±2° C. and at predetermined time periods samples of the dissolution medium are withdrawn to detect the percentage of active ingredient dissolved over time.

The term “gastro resistant”, as used herein can be used synonymously with delayed release.

One embodiment discloses a controlled release composition comprising up to 20% budesonide by total weight of the composition.

Another embodiment discloses a matrix based controlled release composition comprising up to 20% budesonide by total weight of the composition.

Yet another embodiment discloses a controlled release budesonide composition wherein the composition releases not more than about 80% of budesonide within about 8 hours.

Yet another embodiment discloses controlled release composition, wherein the composition is in the form of a tablet, minitablets, pellets, a capsule, a caplet, a sachet, beads or granules.

The amphiphilic excipients which can be used according to the invention may include one or more of polar lipids of type I or II (lecithin, phosphatidylcholine, phosphatidylethanolamine), ceramides, glycol alkyl ethers such as diethylene glycol monomethyl ether.

The hydrophilic excipients include excipients known as hydrogels, i.e. substances which when passing from the dry state to the hydrated one, undergo the so-called “molecular relaxation”, namely a remarkable increase in mass and weight following the coordination of a large number of water molecules by the polar groups present in the polymeric chains of the excipients themselves.

Suitable hydrogels which can be used according to the invention may include one or more of acrylic or methacrylic acid polymers or copolymers, alkylvinyl polymers, hydroxyalkyl celluloses, carboxyalkyl celluloses, polysaccharides, dextrins, pectins, starches and derivatives, natural or synthetic gums, alginic acid. The use of polyalcohols such as xylitol, maltitol and mannitol can also be advantageous in case of taste masking.

Suitable fillers which can be used according to the invention may include one or more of dextrose, sucrose, maltose, and lactose, sugar-alcohols, which include mannitol, sorbitol, maltitol, xylitol, starch hydrolysates, which include dextrins, and maltodextrins, microcrystalline cellulose or other cellulosic derivatives, dicalcium phosphate, tricalcium phosphate, and mixtures thereof and the like.

Suitable binders may include one or more of starch, microcrystalline cellulose, highly dispersed silica, mannitol, lactose, polyvinylpyrrolidone, crosslinked polyvinylpyrrolidone, polymethacrylic acid derivatives, ethyl cellulose, methyl cellulose, hydroxyethyl cellulose cross-linked carboxymethylcellulose, hydroxypropylcellulose, hydroxypropyl methylcellulose and natural and synthetic gums, carbomers, dextrin, zein, gelatin, polymethacrylates, polyvinylpyrrolidone, pregelatinized starch, sodium alginate, gums, synthetic resins and the like.

Suitable disintegrants may include one or more of starch or modified starches, particularly sodium starch glycolate, cornstarch, potato starch or pre-gelatinated starch, clays, particularly bentonite, montmorillonite or veegum; celluloses, particularly microcrystalline cellulose like L-hydroxypropylcellulose or carboxymethylcellulose; alginates, particularly sodium alginate or alginic acid; crosslinked celluloses, particularly croscarmellose sodium; gums, particularly guar gum or xanthan gum; crosslinked polymers, particularly crospovidone and the like.

Suitable lubricants, glidants and antiadherents may include one or more of talc, colloidal silicon dioxide, finely divided silicon dioxide, powdered cellulose, starch, sodium stearyl fumarate, mineral oil, kaolin and the like.

Suitable solubilizers for the purpose of the present invention may include one or more of solubility enhancing agents, dissolution enhancing agents, absorption enhancing agents, penetration enhancing agents, surface active agents and stabilizing agents. The representative, but non-limiting examples of these compounds are Vitamin E TPGS, amino acids such as glutamic acid and glycine, sorbitol, mannose, amylose, maltose, mannitol, lactose, sucrose, glucose, xylitose, dextrins such as maltodextrin, sodium lauryl sulfate, Tween 80 (polyoxyethylene sorbitan monooleate), benzyl alcohol, Poloxamer 407, polyethylene glycols, such as PEG3350; polyvinylpyrrolidones such as PVP K25, polyvinylalcohols, polyalcohols, crospovidone, sodium starch glycolate, croscarmellose sodium, carboxymethylcellulose, starch, pregelatinized starch, HPMC, substituted hydroxypropyl cellulose, microcrystalline cellulose, sodium bicarbonate, calcium citrate and menthol, among others.

Suitable taste-masking agents include, but are not limited to, one or more of polymers, surfactants, sweeteners and flavors. Examples of polymers include one or more of cellulose acetate, polymethacrylates, hydroxypropylmethyl cellulose, hydroxypropyl cellulose, hydroxylethyl cellulose; and the like.

Suitable sweeteners include, but are not limited to, saccharides such as aspartame, sucrose, dextrose, glucose, maltose, dextrins, D-tagatose, trehalose, dried invert sugar, fructose, levulose, galactose, corn syrup solids, and the like, alone or in combination. Other examples of sweeteners comprise sodium saccharin; aspartame; sugarless sweeteners including polyhydric alcohols such as sorbitol, mannitol, xylitol, glycerol, hydrogenated starch hydrolysates, maltitol, isomaltitol, erythritol, lactitol and the like, alone or in combination.

Suitable flavors include, but are not limited to citric acid, cinnamon, wintergreen, eucalyptus, spearmint, peppermint, menthol, anise as well as fruit flavors such as apple, pear, peach, vanilla, strawberry, cherry, apricot, orange, watermelon, banana and the like; bean-derived flavors, such as coffee, cocoa and the like or mixtures thereof.

The composition according to the invention may be subjected to known coating processes with a gastro-resistant film which may comprise one or more of acrylic and methacrylic acids polymers (Eudragit (R)) or copolymer or cellulose derivatives, such as cellulose acetophthalate which include cellulose acetate trimelliate; hydroxypropyl methylcellulose phthalate; hydroxypropyl methylcellulose acetate succinate; polyvinyl acetate phthalate and the like.

The pharmaceutical compositions as described herein may be prepared by processes known to the person having ordinary skill in the art of pharmaceutical technology such as direct compression, wet granulation, dry granulation, melt granulation.

In another embodiment, the method includes a process for providing a controlled release composition, wherein the process includes the steps of:

a) determining the desired release profile,
b) determining specific amounts of budesonide, an amphiphilic excipient, a hydrophilic excipient and one or more pharmaceutically acceptable excipients necessary to produce the pre-determined release profile, and
c) incorporating the specified amounts of the components into the composition.

In one more embodiment, the method comprises a step for providing a controlled release composition, wherein the composition may be prepared by sifting the amphiphilic and hydrophilic excipients with one or more pharmaceutically acceptable excipients followed by mixing with an active ingredient. The obtained mixture may be granulated using a solvent. The granules may be dried and then milled. The dried granules may be blended with one or more pharmaceutically acceptable excipients, lubricated and compressed to obtain final composition, which can further be coated with a delayed release coat.

In another embodiment, the method comprises a step for providing a controlled release composition, wherein the composition may be prepared by sifting an amphiphilic excipient with one or more pharmaceutically acceptable excipients followed by mixing with budesonide. The obtained mixture may be granulated using a solvent. The granules may be dried and then milled. The dried granules may be blended with hydrophilic excipient and other pharmaceutically acceptable excipients, lubricated and compressed to obtain final composition, which can further be coated with a delayed release coat.

In another embodiment, the method comprises a step for providing a controlled release composition, wherein the composition may be prepared by sifting the hydrophilic excipient with other pharmaceutically acceptable excipients followed by mixing with budesonide. The obtained mixture may be granulated using a solvent. The granules may be dried and then milled. The dried granules may be blended with an amphiphilic excipient and one or more pharmaceutically acceptable excipients, lubricated and compressed to obtain final composition, which can further be coated with a delayed release coat.

For the purposes of the present invention, the coating step of the process can be carried out using spraying techniques known in the art or compression coating.

The term “coat” as used herein is defined to mean a coating substantially surrounding a core which provides desirable properties to the dosage form. As is clear to the person of skill in the art, the coat can serve several purposes, including but not limited to protecting the dosage form from environmental conditions, such as light or moisture, providing esthetic or taste-masking properties to the dosage form, making the dosage form easier to swallow or to handle during the production process, or modifying the release properties of the dosage form, such that pharmaceutically active ingredient is released at a different rate from the coated core than from the uncoated core. One or more than one coat, with the same or different functions or properties, can be applied to a core. The term “coat” includes, but is not limited to, modified release coats and non-functional soluble coats.

In another embodiment, the controlled release composition of the present invention may be prepared by the steps comprising:

a) mixing budesonide, an amphiphilic excipient, a hydrophilic excipient and one or more pharmaceutically acceptable excipients,
b) granulating the mixture using a rotary mixer granulator using a solvent,
c) drying the granules obtained,
d) milling the dried granules through a co-mill,
e) lubricating the granules and compressing to obtain tablets; and
f) coating the tablets with a delayed release coat.

In another embodiment, the composition of the present invention comprising budesonide exhibits bioequivalence to a reference composition of budesonide or a pharmaceutically acceptable salt thereof. As used herein, a “reference composition” is intended to mean a composition of budesonide or a pharmaceutically acceptable salt thereof which is currently approved for marketing and which may be used as a reference for a new drug application (NDA) or an abbreviated new drug application (ANDA) under the Federal Food Drug & Cosmetic Act.

Another embodiment discloses a method for the treatment of Inflammatory Bowel Disease or Irritable Bowel Syndrome, comprising administering the controlled release composition of the present invention to a patient in need of such treatment.

The bioequivalence studies were carried out between Uceris® extended release tablets (reference) and compositions of the invention (test) in fasted and fed state. The study was monitored in terms of Cₘₐₓ and AUC achieved with the test product and the reference product (Uceris®).

The invention is further illustrated by the following example which is provided to be exemplary of the invention and does not limit the scope of the invention. While the present invention has been described in terms of its specific embodiments, certain modifications and equivalents will be apparent to those skilled in the art and are intended to be included within the scope of the present invention.

### Example 1

TABLE 1a

| | | |
|---|---|---|
| Sr. | | Quantity |
| No. | Ingredients | (% w/w) |

GRANULATION

| | | |
|---|---|---|
| 1 | Budesonide | 2.77 |
| 2 | Soy Lecithin powder | 2.15 |
| 3 | Lactose monohydrate | 25.86 |
| 4 | Microcrystalline cellulose | 15.39 |
| 5 | Hydroxypropyl cellulose LF | 8.31 |
| 6 | Hydroxypropyl cellulose MXF | 1.54 |
| 7 | Purified water | q.s |

EXTRAGRANULAR

| | | |
|---|---|---|
| 8 | Soy Lecithin powder | 1.54 |
| 9 | Microcrystalline cellulose | 15.39 |
| 10 | Lactose monohydrate | 14.78 |
| 11 | Colloidal silicon dioxide | 0.62 |
| 12 | Sodium stearyl fumarate | 0.92 |

DELAYED RELEASE COATING

| | | |
|---|---|---|
| 13 | Methacrylic Acid Copolymer (type A) | 2.92 |
| 14 | Methacrylic Acid Copolymer NF (type B) | 4.04 |
| 15 | Triethyl Citrate | 0.43 |
| 16 | Talc | 2.12 |
| 17 | Titanium dioxide | 1.21 |
| 18 | Isopropyl alcohol | q.s |
| 19 | Purified water | q.s |

### Process:

Budesonide, soy lecithin powder, lactose monohydrate, microcrystalline cellulose and hydroxypropyl cellulose were sifted and mixed. The mixture was granulated using purified water as solvent. The granules were dried and then milled. The dried granules were blended with soy lecithin powder, lactose monohydrate and microcrystalline cellulose. The blend was lubricated using colloidal silicon dioxide and sodium stearyl fumarate and compressed into tablets using suitable tooling. The tablets obtained were coated with a solution of methacrylic acid copolymers.

### Dissolution Data for Example 1

The dissolution performance was measured using a USP-I rotating basket apparatus. Release times were measured by placing the tablet in a small wire basket placed on the end of a rod spinning at 100 rpm. Aliquots were withdrawn from 0.1 N HCl for 2 hour followed by phosphate buffer pH 7.2 up to 12 hour.

TABLE 1b
Dissolution performance for the final formulation of Example 1

| | Time | % drug |
|---|---|---|
| Medium | (hour) | release |
| 0.1N HCl - | 0 | 0 |
| 500 ml + 0.5% Macrogol | 2 | 0 |
| pH 7.2 Phosphate Buffer - | 1 | 0.8 |
| 1000 ml + 0.5% Macrogol | 2 | 7.5 |
| | 4 | 25.9 |
| | 6 | 50.3 |
| | 8 | 72.6 |
| | 10 | 89.9 |
| | 12 | 96.9 |

### Example 2

TABLE 2a

| | | |
|---|---|---|
| Sr. | | Quantity |
| No. | Ingredients | (% w/w) |

GRANULATION

| | | |
|---|---|---|
| 1 | Budesonide | 2.68 |
| 2 | Soy Lecithin powder | 2.98 |
| 3 | Microcrystalline cellulose | 38.68 |
| 4 | Hydroxypropyl cellulose LF | 8.93 |
| 5 | Purified water | q.s |

EXTRAGRANULAR

| | | |
|---|---|---|
| 6 | Microcrystalline cellulose | 8.33 |
| 7 | Lactose monohydrate | 17.56 |
| 8 | Hydroxypropyl cellulose JXF | 7.14 |
| 9 | Hydroxypropyl cellulose MXF | 1.49 |

LUBRICATION

| | | |
|---|---|---|
| 10 | Colloidal silicon dioxide | 0.60 |
| 11 | Sodium stearyl fumarate | 0.89 |

DELAYED RELEASE COATING

| | | |
|---|---|---|
| 12 | Methacrylic Acid Copolymer (type A) | 4.12 |
| 13 | Methacrylic Acid Copolymer NF (type B) | 3.37 |
| 14 | Triethyl Citrate | 1.07 |
| 15 | Talc | 1.61 |
| 16 | Titanium dioxide | 0.54 |
| 17 | Acetone | q.s |
| 18 | Purified water | q.s |

### Process:

Budesonide, soy lecithin powder, microcrystalline cellulose and hydroxypropyl cellulose were sifted and mixed. The mixture was granulated using purified water as solvent. The granules were dried and then milled. The dried granules were blended with hydroxypropyl cellulose grades, lactose monohydrate and microcrystalline cellulose. The blend was lubricated using colloidal silicon dioxide and sodium stearyl fumarate and compressed into tablets using suitable tooling. The tablets obtained were coated with a solution of methacrylic acid copolymers.

### Dissolution Data for Example 2

The dissolution performance was measured using a USP-I rotating basket apparatus. Release times were measured by placing the tablet in a small wire basket placed on the end of a rod spinning at 100 rpm. Aliquots were withdrawn from 0.1 N HCl for 2 hour followed by phosphate buffer pH 7.2 up to 12 hour.

TABLE 2b
Dissolution performance for the final formulation of Example 2

| | Time | % drug |
|---|---|---|
| Medium | (hour) | release |
| 0.1N HCl - | 0 | 0 |
| 500 ml + 0.5% Macrogol | 2 | 0 |
| pH 7.2 Phosphate Buffer - | 1 | 3.9 |
| 1000 ml + 0.5% Macrogol | 2 | 14.9 |
| | 4 | 38.5 |
| | 6 | 58.2 |
| | 8 | 74.0 |
| | 10 | 86.1 |
| | 12 | 94.0 |

### Bioavailability Study

In-vivo study was conducted in healthy human volunteers to assess bioavailability of budesonide controlled release tablets (Test—composition of the invention as per Example 2) and Uceris® (Reference).

TABLE 2c
Summary of PK parameters of Reference and
Test compositions under Fasting condition

| | REFERENCE | TEST |
|---|---|---|
| | GEOMETRIC | GEOMETRIC |
| PARAMETER | MEANS | MEANS |
| Ln (Cₘₐₓ) (pg/ml) | 2620.20 | 2709.72 |
| Ln (AUCₜ) (pg*h/ml) | 24583.00 | 25242.97 |
| Ln (AUCᵢ)(pg*h/ml) | 25724.00 | 26284.75 |

While the invention has been described in terms of its specific embodiments, certain modifications and equivalents will be apparent to those skilled in the art and are intended to be included within the scope of the invention.

We claim:

1. A controlled release pharmaceutical composition comprising:
(1) a tablet core comprising budesonide in an amount effective for the treatment of inflammatory bowel disease in the gastrointestinal tract, an amphiphilic excipient, a hydrophilic excipient and one or more pharmaceutically acceptable excipients; and
(2) a coating on the tablet core, wherein the coating comprises a gastro-resistant film.

2. The controlled release pharmaceutical composition according to claim 1, wherein the composition comprises about 1 mg to about 12 mg of budesonide.

3. The controlled release pharmaceutical composition according to claim 1, wherein the amphiphilic excipient comprises one or more of polar lipids of type I or II, ceramides, glycol alkyl ethers, esters of fatty acids with polyethylene glycols, and diethylene glycols.

4. The controlled release pharmaceutical composition according to claim 1, wherein the amphiphilic excipient comprises about 1% to about 15% by weight of the composition.

5. The controlled release pharmaceutical composition according to claim 1, wherein the amphiphilic excipient is soy lecithin.

6. The controlled release pharmaceutical composition according to claim 1, wherein the hydrophilic excipient comprises one or more of acrylic or methacrylic acid polymers or copolymers, alkylvinyl polymers, hydroxyalkylcellulose, carboxyalkylcellulose, polysaccharides, dextrins, pectins, starches and derivatives, alginic acid, natural or synthetic gums, and polyalcohols.

7. The controlled release pharmaceutical composition according to claim 1, wherein the hydrophilic excipient comprises about 1% to about 30% by weight of the composition.

8. The controlled release pharmaceutical composition according to claim 1, wherein the hydrophilic excipient is hydroxyl propyl cellulose.

9. The controlled release pharmaceutical composition according to claim 1, wherein the budesonide is homogeneously dispersed in the amphiphilic excipient.

10. The controlled release pharmaceutical composition according to claim 1, wherein the budesonide is homogeneously dispersed both in the hydrophilic excipient and in the amphiphilic excipient.

11. The controlled release pharmaceutical composition according to claim 1, wherein the composition is in the form of a tablet, minitablets, pellets, a capsule, a caplet, a sachet, beads or granules.

12. The controlled release pharmaceutical composition according to claim 1, wherein the composition releases not more than about 80% of budesonide within about 8 hours after administration.

13. The controlled release pharmaceutical composition according to claim 1, wherein the pharmaceutically acceptable excipients comprise one or more of fillers, binders, disintegrants, lubricants, glidants, antiadherents, solubilizers, sweeteners, flavors and taste-masking agents.

14. A process for the preparation of a controlled release pharmaceutical composition comprising budesonide or a pharmaceutically acceptable salt thereof, the process comprising the steps of:
a) mixing budesonide with an amphiphilic excipient, a hydrophilic excipient and one or more other pharmaceutically acceptable excipients;
b) granulating the mixture using a solvent;
c) drying the granules obtained;
d) milling the dried granules through a co-mill;
e) blending the milled granules with the remaining quantity of amphiphilic excipient and one or more pharmaceutically acceptable excipients;
f) lubricating the blend and compressing into tablets; and
g) optionally coating the tablets with a gastro-resistant coat.

15. The controlled release pharmaceutical composition according to claim 1 comprising:
(1) a tablet core comprising budesonide in an amount effective for treatment of inflammatory bowel disease in the gastrointestinal tract, an amphiphilic excipient, a hydrophilic excipient; and one or more pharmaceutically acceptable excipients; and
(2) a gastro-resistant coating on the tablet core,
wherein the composition exhibits no significant difference in both rate and extent of absorption of budesonide as compared to extended release composition of budesonide marketed under the trade name Uceris®.

16. A method for the treatment of Inflammatory Bowel Disease and Irritable Bowel Syndrome comprising administering the controlled release pharmaceutical composition according to claim 1 to a patient in need of such a treatment.
